(19) United States
(12) Patent Application Publication
(10) Pub. No.: US 2003/0156681 A1
(43) Pub. Date: Aug. 21, 2003

(54) Dental imaging system and apparatus using IEEE 1394 protocol

(76) Inventors: Egidio Cianciosi, Scottsdale, AZ (US); Felix Hovsepian, Phoenix, AZ (US)

Correspondence Address: Lawrence R. Oremland, P.C., Suite C-214, 5055 East Broadway Blvd., Tucson, AZ, 85711, US

(21) Appl. No.: 10/366,917

(22) Filed: Feb. 14, 2003

Related U.S. Application Data
(60) Provisional application No. 60/357,327, filed on Feb. 15, 2002.

(51) Int. Cl. A61B 6/14

(52) U.S. Cl. 378/38; 378/168; 378/191

(57) ABSTRACT

A dental imaging system and apparatus, designed for receiving dental image data and transmitting dental image data in accordance with the IEEE 1394 protocol is disclosed. A digital image integration device is configured to (a) receive dental image data from any or all of a plurality of dental image recording devices, each of which is configured to record and output image data, and to (b) transmit digital image data, via a plurality of IEEE 1394 connectors, to any or all of a plurality of digital image receiving devices via the IEEE 1394 protocol At least one of the image recording devices is a single frame image recording device, preferably a filmless radiography sensor. The plurality of dental image recording devices further preferably includes an intraoral video camera configured to record and transmit intraoral video images. In addition, the plurality of digital image receiving devices preferably comprises at least one image display device.

### RELATED APPLICATION/CLAIM OF PRIORITY

This application is related to and claims priority from Provisional Application Serial No. 60/357,327, filed Feb. 15, 2002, which provisional application is incorporated by reference herein.

### FIELD OF THE INVENTION

This invention relates to a dental imaging system and apparatus, and more particularly to a dental imaging system and apparatus configured to receive image data from a plurality of intraoral dental imaging devices and to transmit the image data to various output devices via the IEEE 1394 protocol.

### BACKGROUND

Dentists and oral surgeons typically use x-radiation (“x-rays”) and video to obtain images of their patients' teeth, mouths and gums to aid in diagnosis and treatment. In traditional oral and dental radiography, a cartridge containing a piece of photographic film is placed in the patient's mouth, for example behind a patient's tooth, and an x-ray beam is projected through the tooth and onto the film. The film, after being exposed in this manner, is developed in a dark room or a closed processor using special chemicals to obtain a photographic image of the tooth.

More recently, the field of filmless dental radiography has emerged. In filmless dental radiography, an x-ray beam is still projected through the patient's tooth, but no photographic film is used. Instead, an electronic sensor is placed in the patient's mouth behind the tooth to be examined. The electronic sensor may include a charge-coupled device (CCD), a complementary metal oxide semi conductor (CMOS), or any other filmless radiation sensor. The x-rays pass through the tooth and impinge on the electronic sensor, which converts the x-rays into an electrical signal. The electrical signal is transmitted over a wire to a computer, either directly or though a module containing intermediate processing circuitry. The computer then processes the signal to produce an image on an associated output device, such as a monitor or a printer.

Filmless dental radiography offers several advantages over traditional film-based radiography. Most importantly, the electronic sensor is much more sensitive to x-rays than is film, allowing the dosage of x-rays to the patient to be lowered by as much as 90%. Also, the image of the tooth is generated by the computer almost instantaneously, thus eliminating the entire developing process, including the use of potentially harmful chemicals. In addition, because the images are generated electronically, they can be stored electronically in a computer database. Examples of filmless dental radiography systems include those described in U.S. Pat. No. 4,160,997 to Robert Schwartz and U.S. Pat. No. 5,434,418 to David Schick. Filmless dental radiography systems typically utilize a standard desktop computer, such as an IBM or IBM compatible type personal computer.

Data Path from the Electronic Sensor to Other Devices

PCI and ISA

To provide a data path between the electronic sensor (or the intermediate module) and the computer's CPU, some conventional systems use the computer's Peripheral Component Interconnect (PCI) bus. The PCI bus, a internal 32-bit local bus that runs at 33 MHz and carries data at up to 133 megabytes per second (MBps). Other conventional filmless dental radiography systems use the computer's Industry Standard Architecture (ISA) bus, an 8- or 16-bit internal bus that carries data at up to 8.33 MBps.

While generally good for their intended applications, systems that use the computer's PCI or ISA bus have certain drawbacks. Most notably, the PCI and ISA buses are internal, and require that a specially designed circuit board be installed inside of the computer. Furthermore, the ISA bus is now considered obsolete and can rarely be found in new personal computer systems.

Installing such a board is a time-consuming task that may only be performed by someone trained in the installation of computer peripherals. In particular, the installation requires the physical opening of the computer's housing, the clearing of any casing or wiring that may be in the way of the slot, the insertion of the card into the slot and the re-assembly of the housing once the insertion is complete. These are not tasks that are easily performed by the typical user of a filmless dental radiography system, such as a dentist, endodontist, oral surgeon or any of their clinical staff.

In addition, many practitioners use a single sensor in conjunction with several computers, such as having a separate computer associated with each patient chair in the practitioner's office. For such a scenario to be practical, a separate board must be installed into each of the computers, further increasing the cost of the overall system.

Moreover, the number of PCI and ISA slots available in a desktop or tower computer is limited. Installing a circuit board in a given slot to support a filmless dental radiography system precludes the use of that slot for some other type of peripheral device. Once all slots for a given bus are used, no more peripherals can be interfaced through that bus, unless one of the installed boards is removed and replaced with the board for the new peripheral. Such removal and replacement is not something that can be conveniently done on a regular basis.

USB

Portable personal computers are not available with PCI or ISA slots. Accordingly, a conventional filmless dental radiography system cannot be used with such portable computers, as a result some systems are now available with a Universal Serial Bus (USB) port. The USB is a serial 12 megabits per second (Mbps) channel that can be used for peripherals. Personal computers are now also available with a Universal Serial Bus (USB) port.

The USB is much slower than the PCI or ISA buses. More particularly, the theoretical maximum bandwidth of the USB is 12 Mbps (1.5 MBps), several times slower than the 8.33 MBps ISA bus and orders of magnitude slower than the 133 MBps PCI bus. And because many peripherals might be connected to the USB, no single peripheral can expect to realize the full range of the 1.5 MBps maximum theoretical bandwidth of the USB, making the practical bandwidth of the USB substantially less.

The USB is a token-based bus. In particular, the USB host controller broadcasts tokens on the bus and a device that detects a match on the address in the token responds by either accepting or sending data to the host. The host also manages USB bus power by supporting suspend/resume operations.

Unlike the PCI and ISA buses, the USB port does not require the use of a specially designed circuit board inside the computer. Accordingly, once the appropriate software has been installed, a peripheral simply need be plugged into the USB port to be ready for operation. In addition, one device can be unplugged and another plugged in without changing the hardware configuration of the computer.

Also, the USB port is “hot swappable,” meaning that a first peripheral may be unplugged and a second peripheral plugged in without turning off and restarting the computer. In addition, the USB uses tiered star topology, allowing up to 127 different peripherals on the bus at a time. Further still, not only desktop and tower computers have USB ports; laptop and notebook computers are provided with USB ports as well.

In a conventional filmless dental radiography system analog data might be read-out of the sensor at a rate on the order of 4 million pixels per second (Mpps), converted on a real-time basis to digital data by an analog-to-digital converter (ADC) in an intermediate module and provided on a real-time basis to the computer's PCI or ISA bus. If a 16-bit (2 byte) ADC is used, an interface that can carry data at 8 MBps is required for such data transfer. This is several times greater than even the 1.5 MBps theoretical maximum bandwidth of the USB. Even a system which reads-out data at rate of 1 Mpps and uses a 12-bit (1.5 byte) ADC requires 1.5 MBps of bandwidth, the theoretical maximum bandwidth of the USB, and would strain or exceed the capabilities of the USB. Accordingly, the USB is not believed to be fast enough to support the data flow requirements of a scientific sensor, such as a filmless dental radiography sensor.

One approach is to accommodate the USB bandwidth by simply reading-out data more slowly. This approach, however, is not suitable since a slower readout rate results in a greater accumulation of dark signal (i.e. that part of the image data created by thermally generated electron-hole pairs) in the sensor, which results in turn in greater image degradation. Such results are completely unacceptable for a scientific sensor utilized in a dental radiography system, which must produce images of clarity sufficient to facilitate the diagnosis and treatment of cavities, dental roots and the like.

A dental radiography system that utilized the USB bus port is described in U.S. Pat. No. 6,134,298. This system is only a partial solution since the dentists also wish to connect other peripheral devices, such as video cameras that can capture full-color video at 30 fps (frames per second) at resolutions that exceed 640×480 pixels per frame (e.g. 800×600, 1024×768 and 1280×1024). It is well known that USB is unable to cater for such devices and one must look elsewhere for a solution.

IEEE 1394

IEEE 1394 standard was conceived by Apple Computer and then developed within the IEEE 1394 Working group. This bus supports data transfer rates of 100 Mbps to 3.2 Gbps (Giga Bit Per Second). The standard defines the media, topology and the protocol. The main advantage of the IEEE 1394 bus standard over USB, PCI and others is its speed and the ability to move large amounts of data between computers and peripheral devices.

IEEE 1394 is a digital interface that eliminates the need to convert digital data into analog. Furthermore it is “hot-swappable” meaning devices can be added and removed while the bus is active.

Apple's implementation of the IEEE 1394 is called Firewire.

Computer Bus Ports

Each of these buses may act as a suitable interface between the sensor and computer. However, none of these systems provides a solution where a computer is not available.

As personal computers become smaller and contain fewer expansion slots (PCI, ISA) etc it becomes necessary to find alternate methods to connect external peripherals to these machines.

To make matters more complex there are only one or two “slots” available in a typical portable device where one can connect external peripherals. It is likely that in the future the dentists will have a myriad of devices that they will wish to connect to their portable computers, and a solution that will have the longevity must be found today.

The solution of the present invention combines the video and the filmless dental radiographic system into one bus port, namely the IEEE 1394 port. To date no such solution has been proposed since many of the systems used today use the ISA/PCI or USB bus ports for the filmless dental radiography system and a separate PCI or AGP (Accelerated Graphics Port) for the video capture. PCI/AGP ports are not readily available in notebook/laptop personal computers and a consequence such solutions fails to meet the needs of the dentist.

In addition, a typical dental operatory is 10 ft by 10 ft, with many pieces of furniture and dental equipment. Having a different method to connect the external devices to the personal computer means there are likely to be many cables and other external boxes that simply make the space that the dentist has to work in more cluttered and accident prone, and in general dentists refuse to accept such solutions.

Finally, the solution must also cater for the dentist who wishes to move the external devices between personal computers installed in various operatories within the clinic, whether they are desktop, laptop or other configuration. Therefore the removal and installation of such devices must not necessitate a computer technician, or a lengthy and intricate step-by-step process.

### SUMMARY OF THE PRESENT INVENTION

The present invention provides a new approach to a dental imaging system and apparatus, designed to address the foregoing issues, and to provide a way of receiving dental image data and transmitting dental image data in accordance with the IEEE 1394 protocol. According to the present invention, a digital image integration device is configured to (a) receive dental image data from any or all of a plurality of dental image recording devices, each of which is configured to record and output image data, and to (b) transmit digital image data, via a plurality of IEEE 1394 connectors, to any or all of a plurality of digital image receiving devices via the IEEE 1394 protocol At least one of the image recording devices is a single frame image recording device, preferably a filmless radiography sensor. The plurality of dental image recording devices further preferably includes an intraoral video camera configured to record and transmit intraoral video images. In addition, the plurality of digital image receiving devices preferably comprises at least one image display device.

The present invention enables intraoral video and single frame filmless dental radiographic image recording devices to communicate with a single integration unit that can then be connected to a personal computer via the IEEE 1394 protocol. With the present invention, it is also possible to connect the new unit to a TV (or monitor) where a personal computer is not available. In addition, the IEEE 1394 bus runs typically at 400 Mbps, and is considerably faster then that required to transmit a typical radiographic image read from a sensor. The present invention does not use the IEEE 1394 bus for its speed, but for the fact that visual color video devices can be combined with a dental radiographic device into one unit, in addition to solving some of the pertinent problems with the other bus ports commonly found in personal computers.

Accordingly, an object of the present invention is to provide a way of transmitting single frame filmless dental radiography image data, as well as intraoral video camera image data, in a manner that does not exhibit the disadvantages using the PCI or ISA buses that are discussed at some length above.

Another object of this invention is to provide a way of transmitting single frame filmless dental radiography image data that uses the IEEE 1394 protocol as an interface. In the applicants' experience, this approach is counterintuitive to the ordinary wisdom for transmitting single frame filmless dental radiography image data.

Still another object of the present invention is to transmit mega pixel data, of the type provided by a filmless dental radiography sensor, in a manner that enables dental images of a quality and clarity comparable comparable to that provided in medical environments (with much more powerful and costly equipment) to be produced in a dental operatory environment in a time frame that is particularly useful to dentists and dental staff.

Further features and objectives of the present invention will become apparent from the following detailed description and the accompanying drawings.

### BRIEF DESCRIPTION OF THE DRAWINGS

FIG. 1 is a schematic illustration of a dental imaging system configured according to the principles of the present invention;

FIG. 2 is a schematic illustration of the component parts of a dental image integration device, according to the principles of the present invention;

FIGS. 3-6 illustrate a filmless radiography sensor of a type shown in U.S. application Ser. No. 10/056,419, and which can be used in a system according to the present invention (FIG. 5 is a sectional view of the sensor, taken from the direction 5-5 in FIG. 4); and

FIGS. 7 and 8 illustrate an intraoral video camera, of a type shown in U.S. application Ser. No. 10/005,326, which can be used in a system according to the present invention.

### DETAILED DESCRIPTION

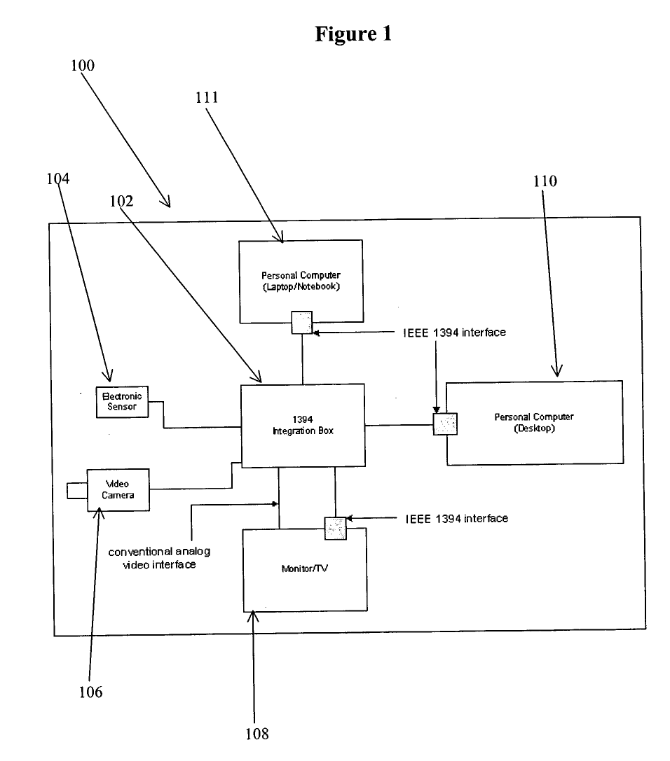

As set forth above, the present invention provides a dental imaging system and apparatus, designed for receiving dental image data and transmitting dental image data in accordance with the IEEE 1394 protocol. FIG. 1 illustrates a dental imaging system 100, with a digital image integration device 102 configured to (a) receive dental image data from any or all of a plurality of dental image recording devices, each of which is configured to record and output image data, and to (b) transmit digital image data, via a plurality of IEEE 1394 connectors, to any or all of a plurality of digital image receiving devices via the IEEE 1394 protocol As illustrated in FIG. 1,at least one image recording device 104 is a single frame image recording device, preferably a filmless radiography sensor. In the system of FIG. 1, another of the dental image recording devices is an intraoral video camera 106 configured to record and transmit intraoral video images. In addition, in the system of FIG. 1, the plurality of digital image receiving devices preferably comprises at least one image display device 108.

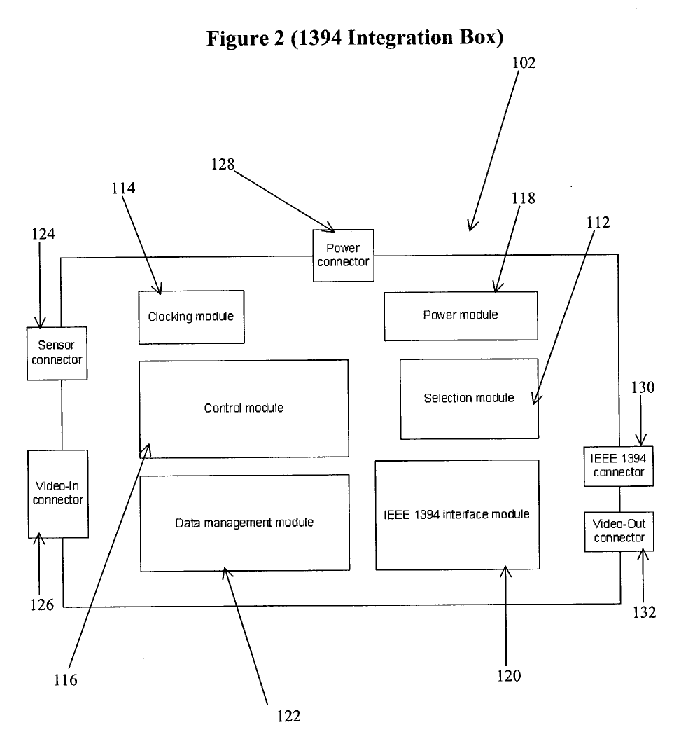

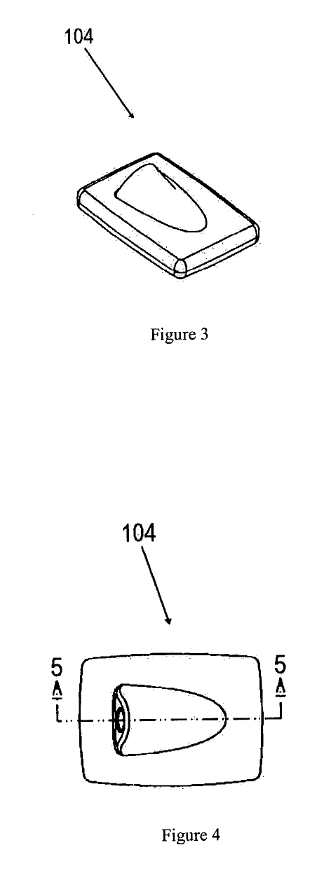

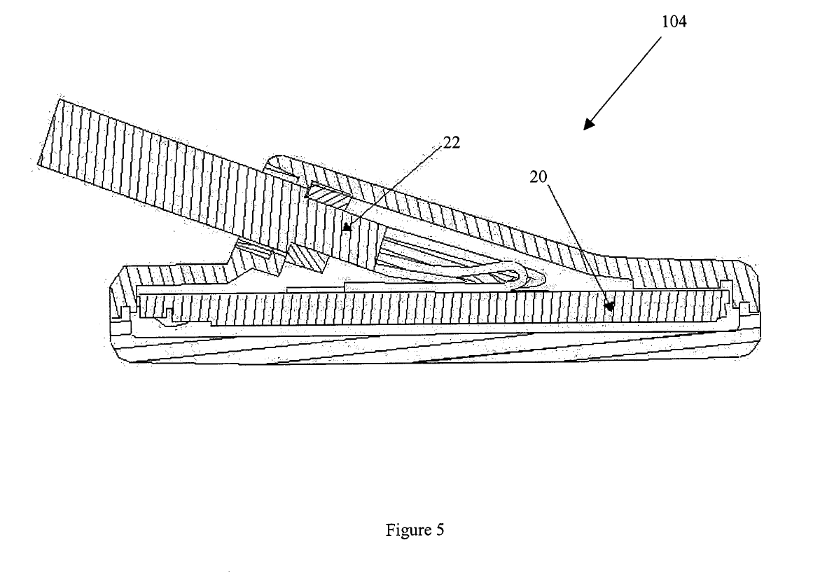

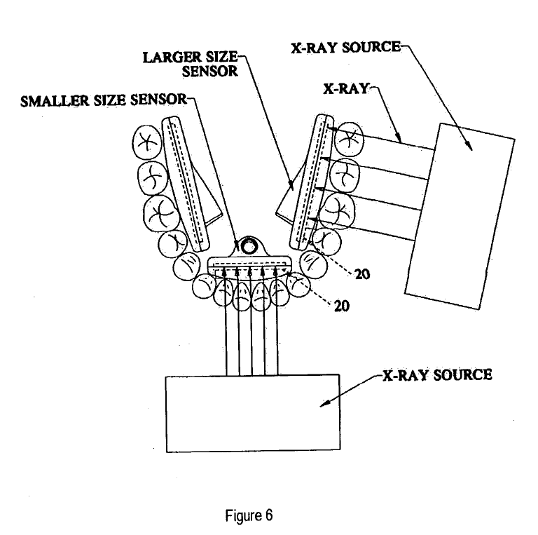

The filmless radiography recording device 104 can take various forms. One example of a filmless radiography recording device which can be used with the present invention is shown and described in application Ser. No. 10/056,419, entitled “Intraoral Sensor”, filed Jan. 24, 2002, by Egidio Cianciosi, the disclosure of which is incorporated by reference herein. FIGS. 3-6 correspond to FIGS. 1, 5, 8 and 10 of that appliction, respectively. The filmless radiography recording device has internal structure, schematically illustrated at 20 in FIG. 5, which is designed in accordance with the principles of U.S. Pat. No. 4,160,997 to Robert Schwartz. The internal structure 20 includes components which receive radiated energy image data from a target area of an intraoral cavity (e.g. x-ray images of a patient's teeth, gums, etc, as shown in FIG. 6), convert the resulting x-ray image data into a visible light image, and transmit the visible light image to a charge coupled device (CCD) or any other visible light sensor forming part of the structure 20. For example, the internal structure 20 could include an array of CCD detectors, a printed circuit board associated with the CCD detectors, a radiant energy screen with a phosphor coating to convert a radiant energy image to a visible image that impinges on the CCD detectors. The printed circuit board is coupled to a cable 22 (FIG. 5) which transmits the image data from the CCD array to the integration box 102 (FIG. 2) as described further below. Again, the internal structure 20 of the sensor can be constructed in various known ways, and should not require further explanation to those in the art.

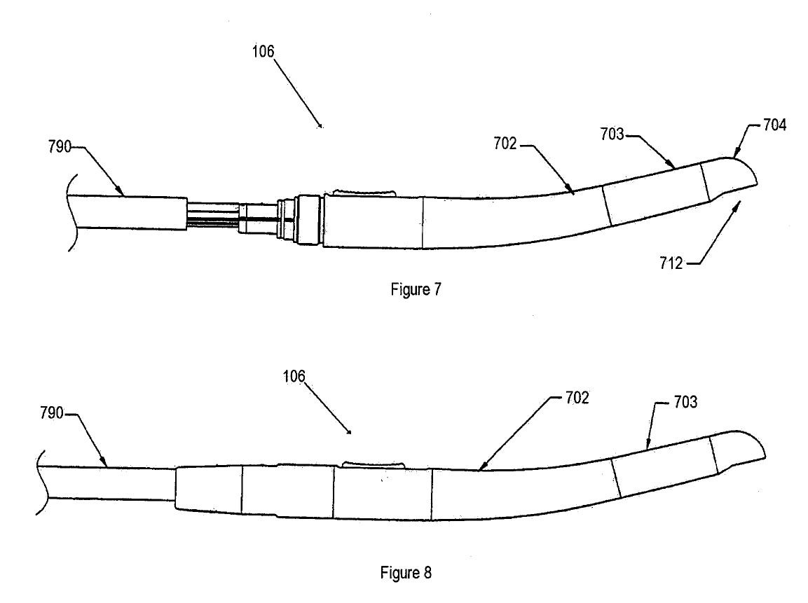

The intraoral video camera 106 can also take various forms, and one example of an intraoral video camera which can be used with the present invention is shown and described in application Ser. No. 10/005,326, entitled “Improved Dental Imaging Apparatus”, filed Nov. 7, 2001, by Egidio Cianciosi, the disclosure of which is incorporated by reference herein. FIGS. 7 and 8 correspond to FIGS. 30 and 31 from that application. The intraoral camera 106 has a bent handle (702) and a headpiece (704) at a distal end of the bent handle. An imaging structure 703 includes an image receptor unit (preferably a CCD array of image receptors) is located adjacent the headpiece 704. The headpiece supports fiber optic bundles that direct light into an intraoral cavity through a viewport 712, and optics (e.g. lens structure in the headpiece 704) is provided for receiving and transmitting viewed images back through the viewport and to the imaging structure. The camera has structure (e.g. a CCD cable located within a cable jacket 790) for transmitting image data from the image receptor unit to an external device, and in accordance with the present invention, that transmitting structure is coupled to the integration box 102, as also discussed below.

The system configuration of the present invention includes the filmless radiography recording device 104, the integration box 102, together with a plurality of output devices. The output devices include the image display device 108, which can be a TV/Monitor and/or personal computer 110 with a processor. If the integration box 102 is connected to a personal computer 110, then the dentist will have full use of the accompanying computer software that will allow the user to capture video and radiographic images, and to save them into an image management database for future reference. Moreover, by connecting the IEEE 1394 integration box 102 to a personal computer the dentist will be able to utilize all of the features that would be available as a software package that would accompany the computer product. Such features could include mirroring, rotating, magnifying the image. In addition, with the IEEE 1394 integration box 102 connected to a personal computer 110 the dentist will be able to save the images captured from the video camera 106, or the filmless dental radiography sensor 104 into an image management database for future reference.

In cases where a personal computer is not available, then the IEEE 1394 integration box 102 can connect directly to the TV/Monitor 108, in which case the user will be able to display a full-color video or a single dental radiographic image. Moreover, the IEEE 1394 integration device 102 can also transmit image data, via the IEEE 1394 protocol, to output devices such as printers, recording devices, etc.

A principal advantage of using the IEEE 1394 integration box 102 of the present invention is that it overcomes many of the difficulties that are inherent with the IS/PCI solutions, in particular this technology allows devices to be “hot-plugged” that is to say external peripherals can be connected and disconnected without first turning the power off on the personal computer. In addition, hubs and repeaters are available for IEEE 1394 bus ports that allow them to be placed conveniently for the dentist, rather than the dentist having to re-organize his work around the personal computer. Finally, up to 64 devices may be simultaneously attached to this kind of port, therefore by using a few of them for the IEEE 1394 integration box does impose a real constraint on the number of other peripherals that could also be used via the 1394 bus port.

The IEEE 1394 Integration Box

The heart of this system is the IEEE 1394 integration box 102. This box consists of the following modules (see FIG. 2):

Selection module 112: This module allows the user/system to select the various features/options that may be incorporated into the integration box at the time of delivery.

Clocking module 114: This contains the circuitry for providing the necessary ‘clocks’ to drive the filmless radiography recording device 104) and/or the intraoral video camera 106. The ‘clocks’ provide the periodic signal that enables the pixels from the electronic sensor (CCD) of the recording device and/or the intraoral video camera 106 to be retrieved into the memory buffers.

Control module 116: Contains the required circuitry to control the filmless radiography recording device 104, analog/IEEE 1394-enabled camera and control of the integration box as a whole. For example, this circuitry provides the functionality that determines the settings on the board so that the clocking module can be initialized.

Power module 118: Provides the power the entire integration box.

IEEE 1394 interface module 120: Provides the functionality for conforming the digital data signals to the IEEE 1394 protocol and for transmitting the digital data signals from the integration box to an output device such as a TV/Monitor.

Data Management module 122: This module is responsible for managing the data retrieved from either filmless radiography recording device 104) and/or the intraoral video camera 106 before it is passed to the IEEE 1394 interface module for transmission to the external display devices.

The IEEE 1394 integration box 102 is configured to retrieve the image, process the image data and output the image data to the personal computer 110 or TV/monitor 108 via a IEEE 1394 bus port. External connectors to the integration box 102 illustrated in the Figures include the following:

Input: Sensor connector 124, for connecting the filmless radiography sensor 104 to the integration box.

Input: Video-in connector 126, for connecting the intraoral video camera 106 to the integration box.

Input: Power connector 128 for supplying the IEEE 1394 integration box 102 with the power necessary to drive the various circuits.

Output: IEEE 1394 connector(s) 130 for connecting the integration box to a (digital) TV/Monitor 108, or to a personal computer 110 or to a laptop computer 111.

Output: Video-out connector 132, for connecting the integration box to a (analog) TV/Monitor.

In the operation of a system according to the present invention, once power is supplied to the integration box 102 the Power module 118 is activated and in turn activates the selection module 112 that allows the user/system to make some configuration choices. The Control module 116 is also activated at the same time. At this time the control module 116 will provide the functionality (for example by using the Clocking module 114, or the Data management module 120) to capture x-rays from the filmless radiography sensor 104, or video from the video camera 106. The control module also controls the IEEE 1394 interface to provide the functionality for conforming the digital data signals to the IEEE 1394 protocol and for transmitting the digital data signals from the integration box 102 to an output device such as a TV/Monitor, thereby providing the essential link to the TV/Monitor, PC (or other output devices).

Thus, as seen from the forgoing description, the present invention provides a new and useful dental imaging system and apparatus, designed for receiving dental image data and transmitting dental image data in accordance with the IEEE 1394 protocol is disclosed. With the foregoing description in mind, the manner in which dental image data can be retrieved from various image recording sources, and transmitted to various output devices via the IEEE 1394 protocol will become apparent to those skilled in the art.

We claim:

1. Apparatus comprising a dental imaging system in which a digital image integration device is configured to
(a) receive dental image data from any or all of a plurality of dental image recording devices, each of which is configured to record and output image data, and to
(b) transmit digital image data, via a plurality of IEEE 1394 connectors, to any or all of a plurality of digital image receiving devices via the IEEE 1394 protocol;
at least one of said image recording devices comprising a single frame image recording device.

2. A dental imaging system as defined in claim 1, wherein said single frame image recording device comprises a filmless radiography sensor, and said plurality of dental image recording devices further comprising an intraoral video camera configured to record and transmit intraoral video images.

3. A dental imaging system as defined in claim 1, wherein said plurality of digital image receiving devices comprises at least one image display device.

4. Apparatus for controlling dental image data, comprising an image integration device configured for connection to a plurality of dental image recording devices including a single frame image recording device and an intraoral video camera configured to record and transmit intraoral video images; said image integration device being further configured to configure and transmit image data from the single frame image recording device and the intraoral video camera in accordance with the IEEE 1394 protocol.

5. Apparatus as defined in claim 4, wherein said integration device comprises an interface module configured to transmit single frame image data retrieved from the single frame image recording device to an external device in accordance with the IEEE 1394 protocol.

6. Apparatus as defined in claim 5, wherein said integration device comprises a control module for controlling the retrieval of single frame image data by said single frame image recording device and the transmission of said single frame image data by the interface module.

7. Apparatus as defined in claim 4, wherein said image integration device is configured to selectively transmit image data in accordance with the IEEE 1394 protocol to any of a plurality of output devices.

8. Apparatus as defined in claim 7, wherein said plurality of output devices includes a digital image viewing device.

9. Apparatus as defined in claim 8, wherein said plurality of output devices further includes a computer processor.

10. Apparatus as defined in claim 9, wherein said computer processor comprises a personal computer processor.

11. Apparatus as defined in claim 9, wherein said processing device comprises a laptop computer processing device.

12. Apparatus as defined in claim 8, wherein said plurality of output devices further includes an image recording device.

13. Apparatus as defined in claim 12, wherein said image recording device comprises an electronic image storage device.

14. Apparatus as defined in claim 8, wherein said plurality of output devices further comprises an electronic printing device.
